(19) United States
(12) Patent Application Publication
(10) Pub. No.: US 2007/0104803 A1
(43) Pub. Date: May 10, 2007

(54) Chinese herbal composition for treating diseases

(75) Inventors: Ing-Jun Chen, Kaohsiung City (TW); Yi-Ching Lo, Kaohsiung City (TW); Yang-Chang Wu, Kaohsiung City (TW); Yi-Hung Tsai, Kaohsiung City (TW); Yung-Hsiung Lai, Kaohsiung City (TW)

Correspondence Address: VOLPE AND KOENIG, P.C., UNITED PLAZA, SUITE 1600, 30 SOUTH 17TH STREET, PHILADELPHIA, PA, 19103, US

(73) Assignee: Kaohsiung Medical University, Kaohsiung City (TW)

(21) Appl. No.: 11/267,791

(22) Filed: Nov. 4, 2005

(51) Int. Cl. A61K 36/539; A61K 36/716

(52) U.S. Cl. 424/725; 424/779; 424/741

(57) ABSTRACT

The present invention relates to a Chinese herbal composition for treating bacterial lipopolysaccharide-induced diseases. The composition is extracted from a mixture of Huang Qui, Huanglian and Dauhuang. The composition is used for treating a lung inflammation and a hypotension.

### FIELD OF THE INVENTION

The present invention relates to a Chinese herbal composition for treating diseases, and more particularly relates to a Chinese herbal composition for treating bacterial lipopolysaccharide-induced diseases.

### BACKGROUND OF THE INVENTION

A sepsis is a systematic inflammation resulting from bacterial infection. The syndrome is followed by septic shock, metabolic acidosis, oliguria, severe hypoxemia. When a patient shows the syndrome of a severe sepsis, his organs have been irreversibly damaged. Finally, the patient will die because of multiple organ failure (MOF). The bacterial infection and non-bacterial infection, such as traumas and acute pancreatitis, will trigger a systemic inflammatory response syndrome (SIRS). In 1991, the American College of Chest Physicians (ACCP) and the Society of Critical Care Medicine (SCCM) define a patient having SIRS if he presents two or more of the following criteria: hyperthermia or hypothermia, tachycardia, tachypnoea and leukocytosis or leukopenia. If a sepsis can not be controlled, a patient will have a severe sepsis. Each organ will lose its function, and this results in a hypoperfusion and a hypotension.

A sepsis starts at a topical infection, such as a pulmonary or abscess of peritoneum. It is infected by bacterial toxins, e.g. endotoxins from the membranes of Gram-negative (G(−)) bacteria, and the fragments of cell walls of Gram-postive (G(+)) bacteria, named exotoxins. Both toxins will trigger macrophages-related cytokines and induce a series of inflammation reactions. The cytokines include tumor necrosis factor α (TNF-α), interleukin-1β (IL-1β) and interleukin-8 (IL-8). The cytokines can be further divided into two groups: one mediates inflammations and the other inhibits the overreaction of inflammations. TNF-α, IL-1, IL-8 and IL-6 are usually as precursors of inflammations. These cytokines will increase the adhesion of white cells in endotheliums, induce the production of proteases and arachidonic acid (AA) and activate the coagulopathy. The arachidonic acid will metabolize to thromboxane A₂ (TXA₂), prostacylin and prostaglandin E₂ (PGE₂), which cause some SIRS-related syndromes, for example, fever, tachycardia, tachypnoea, abnormal of compensation and lactic acidosis. These activated cytokines play important roles for human body against bacterial infections in the early stage, which can activate neutrophils to infiltrate to the affected parts and kill bacteria. However, if these cytokines and the products of bacteria enter the circulation of human body, they will cause a widespread damage of capillaries and result in multiple organ failure.

On the contrary, cytokines for inhibition of inflammation, e.g. IL-10, will affect the feedback regulations of a inflammation and a coagulopathy. In the acute infection stage, these anti-inflammatory cytokines can inhibit the functions of TNF-α, IL-6, T cell-mediated immunoglobulins and macrophage-mediated immunoglobulins, and then activate a compensatory anti-inflammatory response (CARS). If it fails to maintain the balance between SIRS and CARS, then a bad effect would be generated. If the SIRS is dominant, it will show the syndromes of a septic shock or a disseminated intravascular coagulation (DIC). If the CARS is dominant, the immunity of a patient will be inhibited. Consequently, the patient will suffer from bacterial infections and die. Moreover, because of hypoperfusion of main organs, such as kidneys, a brain and livers, it will result in a multiple organ dysfunction (MOD) and is lethal for a patient.

The patho-physiological reaction of a sepsis is divided into an early hyperdynamic state and a late hypodynamic state. In the early hyperdynamic state, it shows the increase of cardiac output and organ perfusion and the decrease of vascular resistance. In the late hypodynamic state, it shows the decrease of cardiac output and organ perfusion. Furthermore, a sepsis causes a dysfunction of endothelia of blood vessels and reduces a production of NO in endothelia. The precursors of inflammations, e.g. TNF-α, also increase in this stage. However, endotoxins may also cause hypotension and brachycardia.

Macrophages are important for a host to be against bacterial or viral infections. When a macrophage is activated by bacterial toxins, e.g. LPS, the macrophage can release some immuno-mediators, such as NO, cytokines and TNF-α, to inhibit the growth of microorganisms. NO is important in various diseases including a sepsis. The production of NO is through the catalysis of L-arginine by NO synthases (NOS). The physiological functions of NO include blood vessel dilatations and neuro-transductions, which serve as an important mediator for a host in defense. There are two kinds of NOS, which are constitutive and inducible. The NOS in endothelia of blood vessels (eNOS) and in neurons (nNOS) are constitutive NOS (cNOS), and their activation is calcium and calmodulin related. Further, the NOS in macrophages, hepatocytes, vascular smooth muscle cells and cardiac myocytes are inducible NOS (iNOS), and their activation is not regulated by cellular calcium. However, iNOS is induced by endotoxins or cytokines, such as LPS, TNF-α and IL-1, and thus iNOS is activated. This results in the mass production of NO.

The arachidonic acid is a 20-carbon fatty acid, which is the main precursor of a prostaglandin. An arachidonic acid is catalyzed by a cyclooxygenase and converted to a eicosanoids, i.e. prostaglandin, for example, PGE₂, TXA₂ and PGI₂, etc. The cyclooxygenase includes a cyclooxygenase-1 (COX-1) and a cyclooxygenase-2 (COX-2). The COX-1 is a constitutive cyclooxygenase, and the COX-2 is induced in an inflammation state.

Under induction by LPS, macrophages can produce several types of cytokines, e.g. tumor necrosis factor-α (TNF-α), interleukin-1 (IL-1β) and IL-6. These cytokines play important roles in human physiology. When macrophages are activated by LPS or cytokines, e.g. TNF-α, genes of inducible nitric oxide synthase (iNOS) and cyclooxygenase-2 (COX-2) will be expressed. Therefore, it will increase the release of PGs and NO. However, it is found in ANA-1 macrophages that PGE₂ induced by LPS can be blocked by an iNOS inhibitor, e.g. aminoguanidine. Therefore, the LPS-induced expression of iNOS is regulated by a prostaglandin. Further, the release of a prostaglandin is self-regulated and induced by the expression of COX-2.

People have used traditional Chinese herbal medicines for thousands of years. Nowadays many reports indicate that lots of traditional Chinese medicines can cure and prevent disease efficiently. It is found that many Chinese herbal medicines have abundant polyphenolic compounds, such as flavonoids, tannins and anthraquinones, which have been proven their ability for anti-inflammation. Huang Qui is widely used in Chinese medicine for anti-bacteria and anti-inflammation. The polyphenols compounds in Huang Qui, anthraquinone derivates in Dauhuang and alkaloid compounds in Huanglian can eliminate free radicals and have anti-inflammation effect. Therefore, it is believed that traditional Chinese herbal medicines could be useful for treating a bacterial induced disease, such as a lung inflammation and a hypotension.

In order to treat a bacterial LPS-induced disease, the present invention provides a new Chinese herbal composition.

### SUMMARY OF THE INVENTION

It is an aspect of the present invention to provide a Chinese herbal composition for treating diseases. The Chinese herbal composition is used for treating bacterial lipopolysaccharide-induced diseases.

According to the present invention, the Chinese herbal composition is extracted from a mixture of Huang Qui, Huanglian and Dauhuang.

Preferably, the mixture of Huang Qui, Huanglian and Dauhuang ratio of 1:1:2 by weight.

More preferably, the Huang Qui is Scutellaria bacicalensis Georgi.

More preferably, the Huanglian is Coptis chinesis Franch.

More preferably, the Dauhuang is Rheum officinale Baill.

According to the present invention, the Chinese herbal composition includes active ingredients of baicalin, baicalein, emodin, wogonin, rhein, berberine, coptisine, palmatine, sennoside A and sennoside B.

According to the present invention, the bacterial lipopolysaccharide-induced disease includes a lung inflammation and a hypotension.

It is another aspect of the present invention to provide a method of preparing a composition for treating a bacterial lipopolysaccharide-induced disease. The method includes the steps of providing a ground powder mixture of Huang Qui, Huanglian, and Dauhuang in a ratio of 1:1:2 by weight, extracting with boiling distilled water for 1 hour, and concentrating the filtrate of extract solution to obtain a semisolid extract, wherein the semisolid extract is then further diluted with distilled water, leading the contents of water in a ratio ranged from 50% to 70% by weight.

Preferably, the Huang Qui is Scutellaria bacicalensis Georgi, the Huanglian is Coptis chinesis Franch, and the Dauhuang is Rheum officinale Baill.

More preferably, the bacterial lipopolysaccharide-induced disease includes a lung inflammation and a hypotension.

It is another aspect of the present invention to provide a method for treating a bacterial lipopolysaccharide-induced disease. The method includes the steps of preparing a composition according to the present invention, and administering the composition to an object in need thereof.

Preferably, the bacterial lipopolysaccharide-induced disease includes a lung inflammation and a hypotension.

It is another aspect of the present invention to provide a physiological active composition including a physiological acceptable carrier and the above-mentioned semisolid extract.

It is another aspect of the present invention to provide a physiological active composition including a physiological acceptable carrier and the Chinese herbal composition according to the present invention.

The above aspects and advantages of the present invention will become more readily apparent to those ordinarily skilled in the art after reviewing the following detailed description and accompanying drawings.

### BRIEF DESCRIPTION OF THE DRAWINGS

FIG. 1 is a diagram showing the HPLC chromatogram of the composition according to the present invention;

FIG. 2 is a diagram showing the effects of the composition according to the present invention on a LPS-induced hypotension;

FIG. 3 is a diagram showing the effects of post-treatment with the composition according to the present invention on a LPS-induced hypotension;

FIG. 4 is a diagram showing the western blot analyses of the effects of the composition according to the present invention on the expression of iNOS induced by LPS;

FIG. 5 is a diagram showing the western blot analyses of the effects of the composition according to the present invention on the expression of COX-2 induced by LPS;

FIG. 6 is a diagram showing the effects of post-treatment with the composition according to the present invention on survival of anaesthetized rats exposed to LPS;

FIG. 7 is a diagram showing the water content of the lungs after administrating LPS and the composition according to the present invention;

FIG. 8 is a diagram showing the inhibitory effects of the composition according to the present invention; and

FIG. 9 is a diagram showing the effects of the composition according to the present invention on the plasma levels of IL-1β, TNF-α and MCP-1.

### DETAILED DESCRIPTION OF THE INVENTION

The present invention will be described more specifically with reference to the following embodiments. It is to be noted that the following descriptions of preferred embodiments of this invention are presented herein for the purpose of illustration and description only; it is not intended to be exhaustive or to be limited to the precise form disclosed.

The crude Chinese herbs, including Huanglian, Huang Qui and Dauhuang, are purchased from Mainland China and supplied by traditional medicine vendors in the Taiwan market. The crude herbal medicines are identified in the Graduate Institute of Natural Product, Kaohsiung Medical University Taiwan, where voucher specimens have been kept. The mixture of Huanglian (Coptis chinesis Franch), root of Huang Qui (Scutellaria bacicalensis Georgi) and rhizome of Dauhuang (Rheum officinale Baill) is collected in an 1:1:2 ratio by weight. The mixture is blended and then extracted with 5 parts distilled water for 1 hour. The solution is centrifuged at 1500×g at room temperature to obtain the supernatant, and then concentrated under reduced pressure at 65° C. to obtain the semisolid form of the Chinese herbal composition according to the present invention, named HBCM thereinafter. The yield rate of the composition, HBCM, is about 23.2%, which is made up by distilled water to contain 60% water.

The water content of the semisolid extract is checked by a moisture analyzer (MF50, AND, Japan). The water extract of HBCM is further diluted with 4 parts distilled water to obtain the working solution for high-performance liquid chromatography (HPLC) (Gilson, France). The working conditions used for the HPLC analysis are as follows: column: Wakosil-II 5C18HG; mobile phase: aqueous phosphoric acid, sodium dodecyl sulfate and acetonitrile; detection: UV, 265 nm; flow-rate: 1.0 ml/min (Okamura et al., 1999). The reference compounds including baicalin, baicalein, emodin, wogonin, rhein, berberine, coptisine, palmatine, sennoside A and sennoside B are purchased from Sigma Chemical Co.

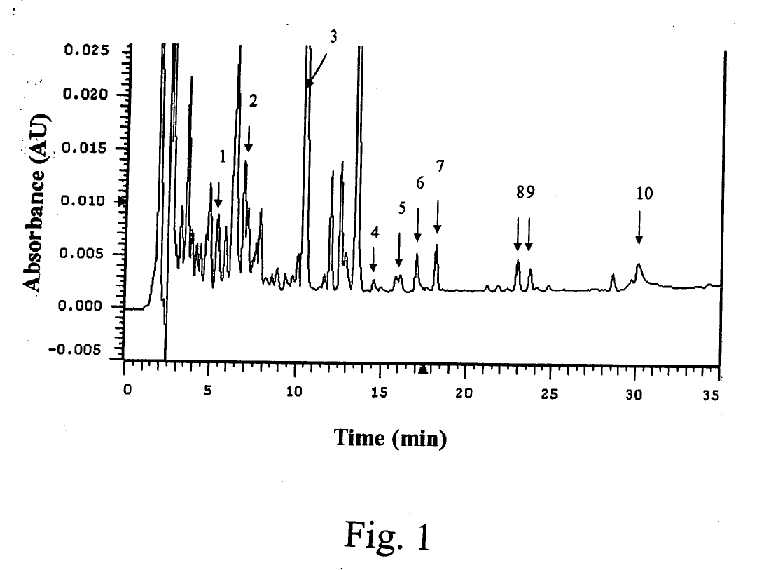

According to the HPLC method, the content (μg/ml) and retention time (Rt, min) of each component in HBCM are as follows: baicalin 1153.07±56.36 (Rt=10.40), baicalein 82.81±3.74 (Rt=18.45), emodin 11.15±1.22 (Rt=24), wogonin 19.55±0.83 (Rt=30.08), rhein 126.12±3.84 (Rt=23.09), berberine 62.14±4.27 (Rt=16.99), coptisine 6.15±0.34 (Rt=14.45), palmatine 25.11±3.78 (Rt=16.05), sennoside A 128.02±13.56 (Rt=7.33) and sennoside B 95.90±3.59 (Rt=5.17). The HPLC trace of HBCM is shown in FIG. 1, in which 1 represents Sennoside B, 2 represents sennoside A, 3 represents baicalin, 4 represents coptisine, 5 represents palmatine, 6 represents berberine, 7 represents baicalein, 8 represents rhein, 9 represents wogonin, and 10 represents emodin. In spite of other unknown chemical components included in HBCM, baicalin is the most abundant one.

The RAW 264.7, a mouse macrophage cell line, is obtained from the American Type Culture Collection (ATCC TIB-71). The cells are cultured in the Dulbecco's modified Eagle's medium (DMEM) supplemented with 2 mM 1-glutamine, antibiotics (100 U/ml of penicillin and 100 U/ml of streptomycin) and 10% heat-inactivated fetal bovine serum (GIBCO/BRL), and maintained at 37° C. in a humidified incubator containing 5% CO₂. The cells in the 2nd-7th passage are used for the experiments.

The animal experiment is approved by the Animal Care and Use Committee at the Kaohsiung Medical University. The male Wistar rats, weighing 250-350 g, are provided by the National Laboratory Animal Breeding and Research Center (Taipei, Taiwan). The rats are housed under conditions of constant temperature and controlled illumination (light regime of 12 h light and 12 h darkness). The rats are allowed food and water ad libitum. They are anesthetized with an intraperitoneal (i.p.) injection of pentobarbital sodium (50 mg/kg). Following tracheal cannulation, systemic arterial blood pressure and heart rate are recorded from the femoral artery with a pressure transducer (Spectramed, Model P10EZ, USA). Body temperature is maintained at 37° C. by an electric heating pad. A femoral vein is cannulated for the administration of LPS (E. coli serotype 026:B6), HBCM, baicalin, or dexamethasone (DEXA), respectively. The rats are injected intravenously with either saline (0.1 ml/kg) or LPS (10 mg/kg) for control and experimental purposes, respectively. All the animals are assigned to one of the following groups: saline only, LPS/saline, LPS/HBCM (75 mg/kg), LPS/baicalin (1.5 mg/kg), and LPS/DEXA (0.5 mg/kg) groups. LPS is injected at a dose of 10 mg/kg, after 10 min, HBCM, baicalin and DEXA are given, respectively.

Experiment 1: Inhibition of LPS-Induced Arterial Hypotension

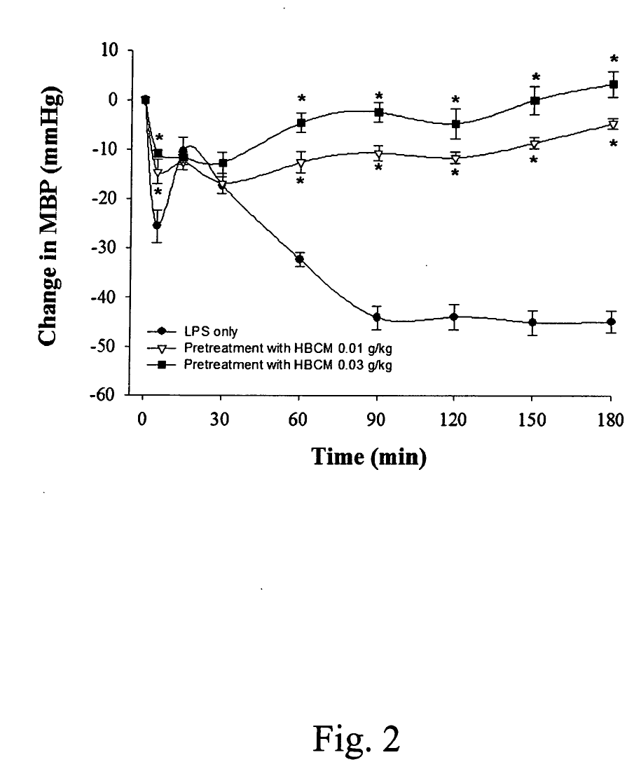

A single injection of LPS (10 mg/kg, i.v.) leads to a biphasic arterial hypotension: an initial and transient decrease in arterial mean blood pressure (about 15 min), followed by a delayed and prolonged hypotension. Rats with normal blood pressure are anesthetized by pentobarbital, and then injected HBCM (0.01 and 0.03 g/kg, i.v.). After 30 minutes, LPS is administrated (10 mg/kg, i.v.). The changes of blood pressure are recorded at 1, 3 and 5 hours after LPS injection. The result is shown in FIG. 2, which demonstrates a dosage-depended increase of heart-beating rates and a prolonged increase of blood pressure.

Experiment 2: Attenuation of LPS-Induced Arterial Hypotension

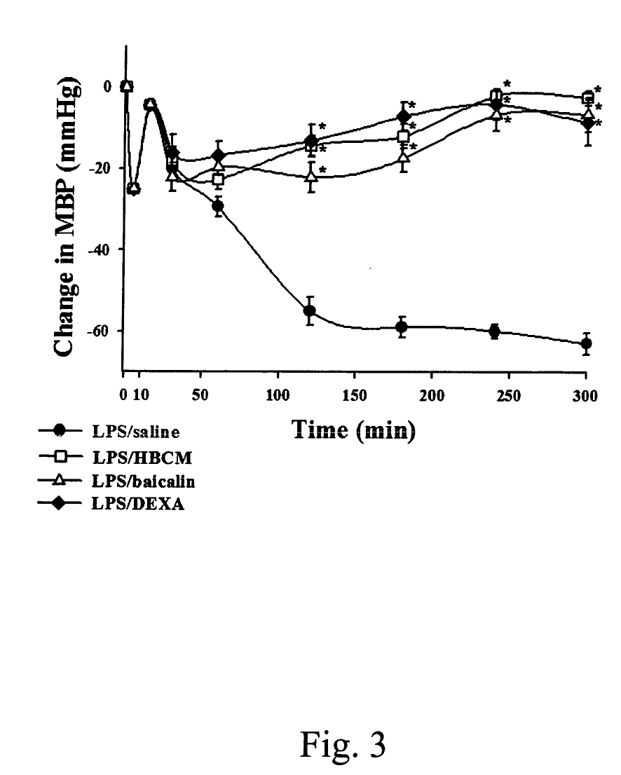

A single injection of LPS (10 mg/kg, i.v.) leads to a biphasic arterial hypotension: an initial and transient decrease in arterial mean blood pressure (about 10 min), followed by a delayed and prolonged hypotension. The HBCM (75 mg/kg, i.v.), baicalin (1.5 mg/kg, i.v.) and DEXA (0.5 mg/kg, i.v.) are administrated 10 min after LPS administration. The used dose of baicalin is according to the content of baicalin in HBCM by the HPLC method. Separate groups are used for acute survival study (n=10 each), lung tissue sampling (n=10 each), and blood sampling (n=10 each). The animals supplied with pulmonary tissues or blood samples are sacrificed under anesthesia. The result shows that the post-treatment with HBCM, baicalin, and DEXA significantly attenuates LPS-induced prolonged arterial hypotension, as shown in FIG. 3.

Experiment 3: Inhibition of LPS-Induced Plasma Cytokines Formation

After LPS (10 mg/kg, i.v.) administration, plasma levels of IL-1β, IL-6, IFN-γ and TNF-α respectively increase in a time-dependent manner. However, pretreatment with HBCM (0.03 g/kg, i.v.) significantly inhibits IL-1β production in the experimental groups treated with LPS for 3 hours. In Table 1, it also illustrates that HBCM (0.03 g/kg, i.v.) significantly inhibits IL-6 production in the experimental groups treated with LPS for 3 and 5 hours. The HBCM (0.01 and 0.03 g/kg, i.v.) also decreases the rise in IFN-γ production in the experimental groups treated with LPS for 3 and 5 hours. In addition, HBCM significantly decreases the rise in TNF-α production in the experimental groups treated with LPS for 3 and 5 hours.

Experiment 4: Inhibition of LPS-Induced Plasma PGE₂ Production

As shown in Table 1, the plasma concentration of PGE2 can be raised significantly in a time-dependent manner after treatment with LPS (10 mg/kg, i.v.) for 3 and 5 hours in Wistar rats. The HBCM (0.01 and 0.03 g/kg, i.v.) blocks the rise in PGE₂ production after LPS treatments for 3 and 5 hours.

TABLE 1

| | 3 h | 5 h |
|---|---|---|
| | (pg/ml) | (pg/ml) |
| IL-1β | | |
| Basal | 90.0 ± 4.1 | 90.0 ± 4.1 |
| LPS only | 373.4 ± 16.0 | 551.1 ± 11.7 |
| LPS + HBCM 0.01 g/kg | 330.4 ± 14.1 | 509.3 ± 30.6 |
| LPS + HBCM 0.03 g/kg | 212.5 ± 18.0* | 495.0 ± 3.5 |
| IL-6 | | |
| Basal | 649.6 ± 43.1. | 649.6 ± 43.1 |
| LPS only | 6823.5 ± 123.7 | 7641 ± 254.0 |
| LPS + HBCM 0.01 g/kg | 5944.5 ± 115.0 | 6680.6 ± 280.3 |
| LPS + HBCM 0.03 g/kg | 5273.5 ± 126.0* | 5500.1 ± 306.2* |
| INF-γ | | |
| Basal | 13.2 ± 1.3 | 13.2 ± 1.3 |
| LPS only | 258.4 ± 14.7 | 331.3 ± 16.2 |
| LPS + HBCM 0.01 g/kg | 153.4 ± 5.6* | 137.4 ± 1.5* |
| LPS + HBCM 0.03 g/kg | 52.6 ± 2.2* | 55.8 ± 1.5* |
| TNF-α | | |
| Basal | 90.0 ± 5.2 | 90.0 ± 5.2 |
| LPS only | 374.0 ± 21.9 | 1789.2 ± 111.3 |
| LPS + HBCM 0.01 g/kg | 216.6 ± 18.3* | 1302.8 ± 94.3* |
| LPS + HBCM 0.03 g/kg | 137.4 ± 5.7* | 1279.5 ± 17.7* |
| PGE₂ | (ng/ml) | (ng/ml) |
| Basal | 7.91. ± 1.4 | 7.91 ± 1.4 |
| LPS only | 51.3. ± 4.2 | 73.8 ± 4.1 |
| LPS + HBCM 0.01 g/kg | 36.5. ± 2.3* | 49.1 ± 3.1* |
| LPS + HBCM 0.03 g/kg | 19.6. ± 1.2* | 35.5 ± 2.6* |

*Significantly different from LPS only, p < 0.05, ANOVA followed by Dunnett's test.

Experiment 5: Inhibition of LPS-Induced Cytokines Formation in RAW 264.7 Macrophages

As illustrated in Table 2, LPS (1 μg/ml) treatment alone causes a marked up-regulation of levels of IL-1β, IL-6, IFN-γ and TNF-α in cell supernatants at 6 and 15 hours. However, HBCM inhibits this induction of IL-1β by LPS at 6 and 15 hours. The pretreatment with HBCM working solution also significantly inhibits the IL-6 production at 6 and 15 hours in a dose-dependent manner. LPS-treated cells show a marked up-regulation of IFN-γ production, and are inhibited by pretreatment with HBCM in a dose-dependent manner. Finally, as shown in Table 2, HBCM inhibits TNF-α production at 6 and 15 hours post-LPS injection.

TABLE 2

| | 6 h (pg/ml) | 15 h (pg/ml) |
|---|---|---|
| IL-1 β | | |
| Basal | 226.6 ± 3.3 | 226.6 ± 3.3 |
| LPS only | 1376.6 ± 50.5 | 2368.9 ± 75.2 |
| LPS + HBCM 20 μg/mL | 1276.2 ± 40.7* | 2200.2 ± 32.6* |
| LPS + HBCM 40 μg/mL | 1200.1 ± 40.5* | 2100.5 ± 50.3* |
| LPS + HBCM 60 μg/mL | 642.5 ± 30.3* | 1900.2 ± 87.0* |
| LPS + HBCM 80 μg/mL | 576.62 ± 20.8* | 1557.3 ± 60.1* |
| LPS + HBCM 100 μg/mL | 450.6 ± 26.5* | 965.1 ± 60.2* |
| LPS + HBCM 200 μg/mL | 430.5 ± 32.6* | 311.2 ± 21.8* |
| IL-6 | | |
| Basal | 195.6 ± 15.3 | 195.6 ± 15.3 |
| LPS only | 5807.2 ± 75.7 | 1767.9 ± 60.2 |
| LPS + HBCM 20 μg/mL | 1809.0 ± 26.5 | 1148.2 ± 58.2 |
| LPS + HBCM 40 μg/mL | 1435.7 ± 30.2 | 1098.2 ± 50.1 |
| LPS + HBCM 60 μg/mL | 1435.7 ± 50.6* | 757.2 ± 60.3* |
| LPS + HBCM 80 μg/mL | 794.7 ± 32.7* | 601.8 ± 16.4* |
| LPS + HBCM 100 μg/mL | 710.7 ± 26.1* | 394.7 ± 18.3* |
| LPS + HBCM 200 μg/mL | 241.1 ± 14.7* | 100.1 ± 8.1* |
| INF-γ | | |
| Basal | 870.2 ± 39.4 | 870.2 ± 39.4 |
| LPS only | 3141.7 ± 112.1 | 3756.0 ± 100.2 |
| LPS + HBCM 20 μg/mL | 2934.5 ± 136.9 | 1270.2 ± 65.2* |
| LPS + HBCM 40 μg/mL | 1770.2 ± 95.6* | 1198.8 ± 86.0* |
| LPS + HBCM 60 μg/mL | 1534.5 ± 90.8* | 1152.4 ± 65.3* |
| LPS + HBCM 80 μg/mL | 1398.8 ± 61.3* | 1113.1 ± 52.1* |
| LPS + HBCM 100 μg/mL | 1200.8 ± 42.4* | 1000.7 ± 52.5* |
| LPS + HBCM 200 μg/mL | 1000.3 ± 32.0* | 856.0 ± 46.7* |
| TNF-α | | |
| Basal | 23.5 ± 7.0 | 23.5 ± 7.0 |
| LPS only | 4784.3 ± 60.2 | 98.1 ± 5.0 |
| LPS + HBCM 20 μg/mL | 4577.6 ± 55.3 | 96.0 ± 2.1 |
| LPS + HBCM 40 μg/mL | 3404.3 ± 60.2* | 80.1 ± 6.0* |
| LPS + HBCM 60 μg/mL | 1762.6 ± 60.3* | 70.0 ± 5.0* |
| LPS + HBCM 80 μg/mL | 1487.6 ± 28.2* | 66.4 ± 4.6* |
| LPS + HBCM 100 μg/mL | 196.0 ± 11.5* | 50.3 ± 4.7* |
| LPS + HBCM 200 μg/mL | 55.3 ± 21.0* | 20.3 ± 2.0* |

*Significantly different from LPS only, P < 0.05, ANOVA followed by Dunnett's test.

Experiment 6: Cell Viability Assay

The RAW 264.7 cells are seeded in 24-well plates at a density of 5×10⁵ cells/ml for each well. After overnight growth, cells are treated with a different concentration of HBCM (20-200 μg/ml) for 6 and 15 hours. After 6 and 15 hours, culture medium from the well is harvested and centrifuged for 1 min. at 12,000×g to remove particles and cells before being divided into 100 μl aliquots for analysis. The samples for lactate dehydrogenase (LDH) assay are stored for 24 hours at 4° C. before use. A commercial LDH assay kit (Roche, U.S.A.) is used to determine cell toxicity. Each sample is tested in triplicate.

According to the result of cell viability assay, it shows that the HBCM is not cytotoxic at the concentration of 20 to 200 μg/ml.

Experiment 7: Inhibition of LPS-Induced iNOS and COX-2 Expression by Western Blot Analysis

RAW 264.7 cells are cultured in a 100 mm plate in the presence of LPS (1 μg/ml) and HBCM (20-200 μg/ml) for 6 hours and 15 hours. Culture media are collected at 6 and 15 hours. Cytokines (IL-1β, IL-6, IFN-γ and TNF-α) are determined using ELISA kits (Endogen, U.S.A.). Each sample is tested in triplicate.

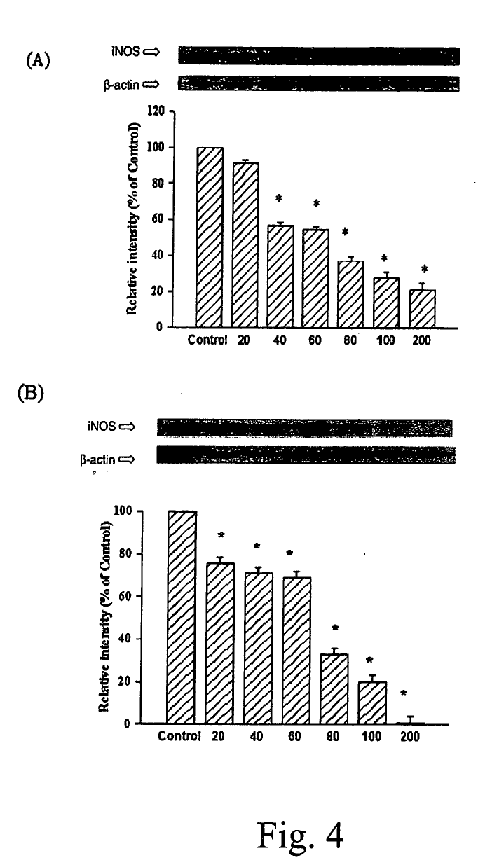

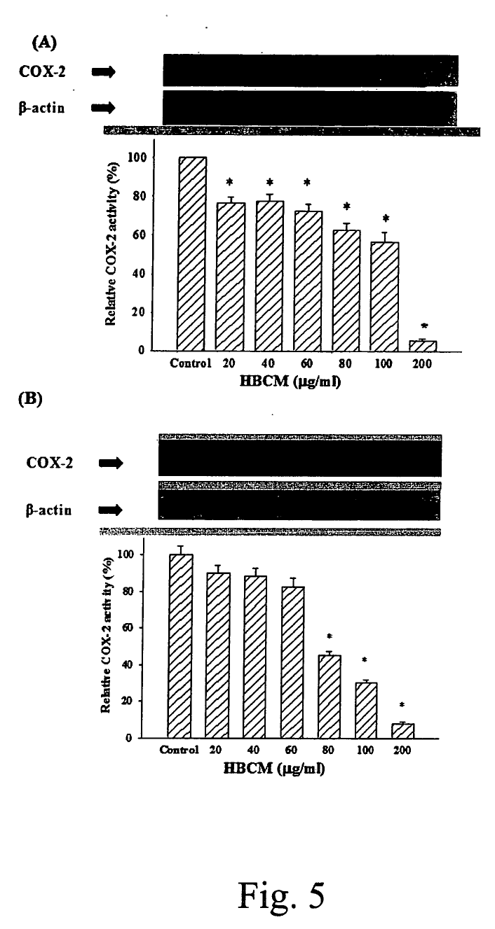

The examination of the cytotoxicity of HBCM in RAW 264.7 macrophages by LDH assay indicates that HBCM, even at 200 μg, does not affect the viability of RAW 264.7 cells (data not shown). Therefore, the inhibition of LPS-induced iNOS and COX-2 protein expression is not the result of a possible cytotoxic effect on these cells. The RAW 264.7 cells do not express detectable iNOS protein when they are incubated in the medium without LPS for 6 and 15 hours. Upon LPS (1 μg/ml) treatment for 6 and 15 hours, the expression of iNOS protein drastically increases in these cells. Co-treatment of cells with LPS (1 μg/ml) and HBCM (20-200 μg/ml) for 6 and 15 hours significantly inhibits iNOS (FIG. 4) and COX-2 (FIG. 5) protein induction in RAW 264.7 macrophages. The amount of β-actin protein as an internal control remains unchanged.

Experiment 8: Acute Survival Studies

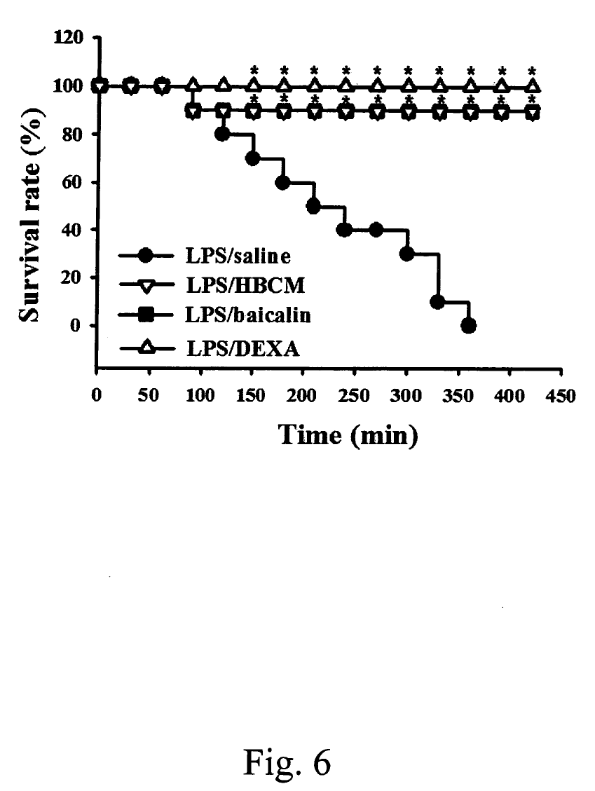

The animals without pulmonary are monitored per 30 minutes for 7 hours after LPS administration. The blood pressure (BP) changes were recorded within 5 hours after LPS injection. Ten minutes after LPS (10 mg/kg) administration, HBCM, baicalin and DEXA are intravenously injected, respectively. In the LPS/saline group, 10% survives for 5 hours and none survives more than 5.5 hours. In both of the LPS/HBCM group and LPS/baicalin group, 90% survives for 7 hours. In the LPS/DEXA group, all the rats survive more than 7 hours. All of the LPS/HBCM group, LPS/baicalin group and LPS/DEXA group show significantly better survival rate than the LPS/saline group (FIG. 6).

Experiment 9: Therapeutic Effects in LPS-Induced Lung Edema

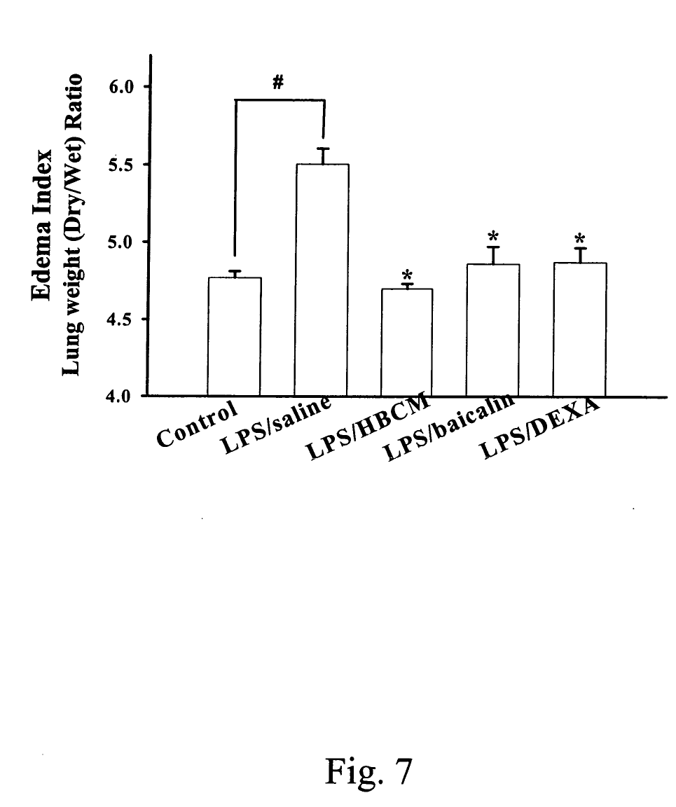

The water content of the lungs is determined by calculating the wet/dry weight ratio of lung tissues at 5 hours after LPS administration. 10 minutes after intravenous administration with LPS, HBCM, baicalin and DEXA are given, respectively. The left lung is dissected free from non-pulmonary tissues, rinsed free from blood, and weighed (wet weight). The dry weight is determined after the lung is dried at 80° C. for 72 hours, and the edema index (wet/dry weight ratio) is calculated by dividing the wet weight by the dry weight. The edema index indicates the water content of the lung, which is determined by calculating the wet/dry weight ratio of lung tissues at 5 hours after LPS administration. Lung wet/dry weight ratios are significantly higher at 5 hours after LPS administration in the LPS/saline group compared with the control (saline only) group. The administration of HBCM, baicalin and DEXA significantly attenuates LPS-induced lung edema (FIG. 7).

Experiment 10: Inhibited LPS-Induced iNOS, Phosphorylated p38 MAP Kinase, TGF-β and ICAM-1 in Lung Tissues

The HBCM, baicalin and DEXA are post-treated 10 minutes after the administration of LPS. The right middle and accessory lobes of the lung are harvested at 5 hours after LPS administration, cleared of non-pulmonary tissues, and frozen at −80° C. until the homogenization. The tissue protein extraction reagent is used and the lysate is centrifuged at 15,000×g for 30 minutes. Then supernatant is freeze-dried. A 8% SDS-polyacrylamide minigels is used, and transferred to immobilon polyvinylidene difluoride membranes (Millipore, Germany). The membrane is incubated overnight at 4° C. with 1% BSA and then incubated with anti-iNOS, anti-TGF-β, anti-ICAM-1, anti-Pp38 MAP kinase and anti-β-actin antibodies. The expression of each protein is detected by enhanced chemiluminescence using Hyperfilm and ECL reagent (Amersham, UK).

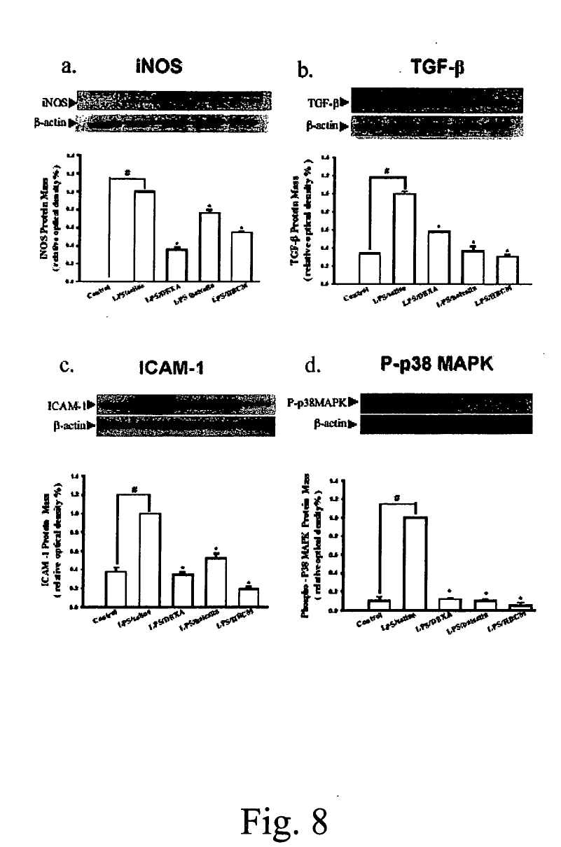

In the LPS group (10 mg kg⁻¹), the expressions of iNOS, phosphorylated p38 MAP kinase, TGF-β and ICAM-1 are all significantly increased than the control (saline) group in the lung tissues, respectively. However, the HBCM (75 mg kg⁻¹), baicalin (1.5 mg kg⁻¹) and DEXA (0.5 mg kg⁻¹) groups show the significant inhibition of LPS-induced phosphorylated iNOS, p38 MAP kinase, TGF-β and ICAM-1 expressions in the lung tissues, respectively (FIG. 8).

Experiment 11: Inhibition of LPS-Induced Plasma Cytokine Immunoreactivities

The blood is collected from a venous cannula, injected into ice-cold heparinized Eppendorf tubes and centrifuged at 1500 rpm for 10 minutes at 4° C. The plasma supernatant is stored at −80° C. until analysis. The assay is carried out by solid phase sandwich enzyme-linked immunosorbent assay (ELISA) kits that specifically detecte IL-1β, TNF-α and MCP-1.

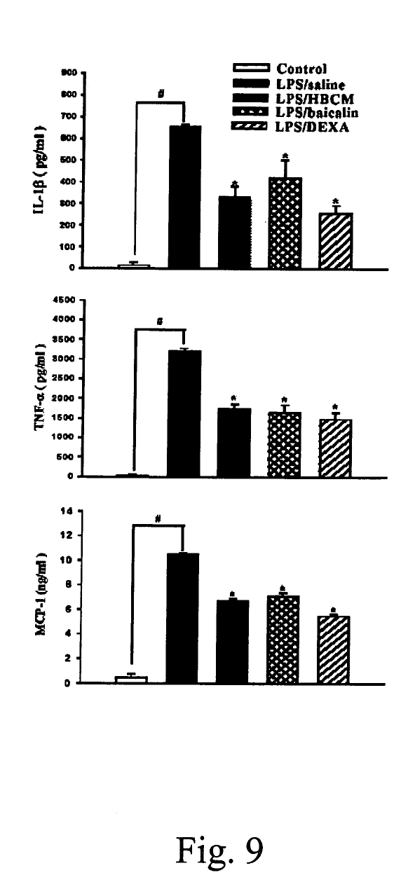

In the LPS/saline group, LPS administration (10 mg kg⁻¹, i.v.) markedly elevates plasma TNF-α concentration within the first 2 hours than the control (saline) group. The HBCM, baicalin and DEXA are post-treated 10 minutes after the LPS administration. As shown in FIG. 9, the elevations of TNF-α in the HBCM (75 mg kg⁻¹), baicalin (1.5 mg kg⁻¹) and DEXA (0.5 mg kg⁻¹) groups are significantly lower than the LPS/saline group. Moreover, the plasma concentrations of IL-1β and MCP-1 increase with time up to 5 hours after LPS administration. The elevations of IL-1β and MCP-1 are lower in the LPS/HBCM, LPS/baicalin and LPS/DEXA groups, compared with the LPS/saline group (FIG. 9).

All of the above-mentioned experiments illustrate the inhibitory and curative effect of HBCM, which is a Chinese herbal composition according to the present invention, for treating a bacterial LPS-induced disease, especially for treating a bacterial LPS-induced lung inflammation and a hypotension.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar formulations.

What is claimed is:

1. A composition extracted from a mixture of Huang Qui, Huanglian and Dauhuang for treating a disease, wherein said Huang Qui, said Huanglian and said Dauhuang are in a ratio of 1:1:2 by weight.

2. The composition according to claim 1 being further diluted with 4˜8 times distilled water to prepare a water soluble solution, leading the contents of water in a ratio ranged from 50% to 70% by weight.

3. The composition according to claim 1, wherein said Huang Qui is Scutellaria bacicalensis Georgi.

4. The composition according to claim 1, wherein said Huanglian is Coptis chinesis Franch.

5. The composition according to claim 1, wherein said Dauhuang is Rheum officinale Baill.

6. The composition according to claim 1, wherein said composition comprises active ingredients of baicalin, baicalein, emodin, wogonin, rhein, berberine, coptisine, palmatine, sennoside A and sennoside B.

7. The composition according to claim 1, wherein said disease is a bacterial lipopolysaccharide-induced disease.

8. The composition according to claim 7, wherein said bacterial lipopolysaccharide-induced disease comprises a lung inflammation and a hypotension.

9. A method for preparing a composition for treating a bacterial lipopolysaccharide-induced disease, comprising:
providing a mixture of Huang Qui, Huanglian, and Dauhuang in a ratio of 1:1:2 by weight;
immersing said mixture with distilled water to form a liquor;
collecting a supernatant of said liquor at room temperature; and
concentrating said supernatant to obtain a semisolid extract, wherein said semisolid extract has distilled water in a ratio ranged from 50% to 70% by weight.

10. The method according to claim 9, wherein said Huang Qui is Scutellaria bacicalensis Georgi.

11. The method according to claim 9, wherein said Huanglian is Coptis chinesis Franch.

12. The method according to claim 9, wherein said Dauhuang is Rheum officinale Baill.

13. The method according to claim 9, wherein said bacterial lipopolysaccharide-induced disease comprises a lung inflammation and a hypotension.

14. A method for treating a bacterial lipopolysaccharide-induced disease, comprising:
preparing a composition according to claim 1; and
administering said composition to an object in need thereof.

15. The method according to claim 14, wherein said bacterial lipopolysaccharide-induced disease comprises a lung inflammation and a hypotension.

16. A physiological active composition comprising a physiological acceptable carrier and said semisolid extract according to claim 9.

17. A physiological active composition comprising a physiological acceptable carrier and said composition according to claim 1.
